(19) United States
(12) Patent Application Publication
(10) Pub. No.: US 2025/0144630 A1
(43) Pub. Date: May 8, 2025

(54) BUFFER EXCHANGE OF BIOLOGICAL SAMPLES IN-LINE WITH SEPARATION AND MEASUREMENT OPERATIONS

(71) Applicants: Nathan SWAMI, Charlottesville, VA (US); Xuhai HUANG, Waynesboro, VA (US); Walter VARHUE, Glen Allen, VA (US); Karina Torres CASTRO, Houston, TX (US); University of Virginia Patent Foundation, Charlottesville, VA (US)

(72) Inventors: Nathan Swami, Charlottesville, VA (US); XuHai Huang, Waynesboro, VA (US); Walter Varhue, Glen Allen, VA (US); Karina Torres Castro, Germantown, MD (US)

(21) Appl. No.: 18/837,107

(22) PCT Filed: Feb. 10, 2023
(86) PCT No.: PCT/US2023/062399

§ 371 Date: Aug. 8, 2024

Related U.S. Application Data
(60) Provisional application No. 63/308,717, filed on Feb. 10, 2022.

(51) Int. Cl. B01L 3/00

(52) U.S. Cl. CPC B01L 3/502776; B01L 3/502761; B01L 2200/0636; B01L 2200/0647; B01L 2300/0883; B01L 2400/0424

(57) ABSTRACT

A method can include receiving a biological sample including the target cells, e.g., at an inlet of a microfluidic structure. This can involve flowing the target cells, e.g., suspended in a first buffer, at a first flow rate within a main channel of the microfluidic structure. At least one focusing flow can be applied to the biological sample, using a second buffer at a second flow rate, to help establish a boundary defining a central region or streamline. The central region can contain the target cells within the main channel and promote ion diffusion between the first and second buffers.

### CLAIM OF PRIORITY

This application claims priority to U.S. Provisional Application Ser. No. 63/308,717, filed on Feb. 10, 2022, which is incorporated by reference herein in its entirety, and the benefit of priority of which is claimed herein.

### STATEMENT REGARDING FEDERALLY SPONSORED RESEARCH OR DEVELOPMENT

This invention was made with government support under Grant No. CA044579, awarded by the National Institutes of Health, Grant No. 2051652, awarded by the National Science Foundation, and Grant No. FA2386-21-1-4070, awarded by the Air Force Office of Scientific Research. The government has certain rights in the invention.

### BACKGROUND

Dielectrophoresis (DEP) is an electrokinetic phenomenon by which particles suspended in a dielectric medium are subjected to a force when exposed to a spatially non-uniform electric field. This force can be used for a variety of applications, such as trapping, sorting, or manipulating particles. Generally, particles are suspended within a buffer solution, and a high-frequency electric field is applied to the buffer solution. In DEP, particles respond differently to different electric fields. For example, particles having a higher dielectric constant than the surrounding medium experience a force in the direction of the electric field gradient, while particles having a lower dielectric constant than the surrounding medium experience a force in the opposite direction.

### SUMMARY

This document describes on-chip sample preparation for biological separation or measurement of cells, such as via dielectrophoresis or impedance methods. Such sample preparation can be performed, e.g., in-line with on-chip monitoring of the outlet sample for metrics of media conductivity, cell velocity, cell viability, cell position, and collected cell numbers. This can help facilitate dielectrophoretic separations from heterogeneous samples. Such on-chip sample preparation can involve a method for exchanging a buffer in which a heterogeneous set of target cells included in a biological sample are suspended, such as for an on-chip biological separation and measurement task. For example, the method can include receiving a biological sample including the target cells, e.g., at an inlet of a microfluidic structure. This can involve flowing the target cells, e.g., suspended in a first buffer, at a first flow rate within a main channel of the microfluidic structure. A set of interfacial focusing flows can be applied to the biological sample, e.g., using a second buffer at a second flow rate, to help establish a boundary defining a central region or streamline. Here, the central region can help contain the target cells within the main channel and help promote ion diffusion between the first buffer and the second buffer. The method can also include performing electrical measurement or electrical separation of the central region of target cells using at least one of electrophoresis, dielectrophoresis, electrochemical measurement, or impedance measurement of cells or media.

In an example, a velocity gradient established at the boundary can help inhibit dispersion of the target cells away from the central region and can support downstream biological separation and measurement of the target cells. For example, the method can include establishing or adjusting a velocity gradient between the first flow rate and the second flow rate to inhibit dispersion of the target cells away from the central region. In an example, the second flow rate can be established at a ratio between 3:1 and 10:1 relative to the first flow rate. The velocity gradient can help suppress or inhibit bulk convection between the first buffer and the second buffer during ion diffusion in the channel of the microfluidic structure. In an example, a flow resistance can be introduced at or near an outlet of the main channel such as to help control a velocity of the target cells in the central region before a downstream operation.

In an example, the microfluidic structure can define respective nozzle structures feeding the main channel. Here, the respective nozzle structures can include a tapered profiled to establish the focusing flow in the main channel. The microfluidic structure can also define side effluent channels to help divert a flow of buffer solution away from the central region at or near the output of the microfluidic structure.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an example of a microfluidic structure for exchanging a buffer of target cells such as for an on-chip biological separation and measurement.

FIG. 1B is a detailed view of the inlet of the main channel of a microfluidic device.

FIG. 1C is another detailed view of the inlet of the main channel of a microfluidic device

FIG. 2A depicts an example of a microfluidic device for exchanging a buffer of target cells such as for on-chip biological separation and measurement.

FIG. 2B is a detailed view of the inlet of the main channel of a microfluidic device.

FIG. 3A depicts another example of a microfluidic device for exchanging a buffer of target cells such as for on-chip biological separation and measurement.

FIG. 3B is a detailed view of the outlet of the main channel of a microfluidic device.

FIG. 3C shows a chart depicting an example of a modeling exercise used to calculate the desired length of the microfluidic device.

FIG. 4A shows a connection from buffer swap region to a DEP device region through a serpentine channel to modulate hydrodynamic resistance.

FIG. 4B depicts an example of a microfluidic device for separating particles in a sample based on biophysical properties.

FIG. 4C depicts an example of a microfluidic device for separating particles in a sample based on biophysical properties.

FIG. 4D depicts an electrical separation or electrical sensing DEP device.

FIG. 4E depicts an expanded view of the orifice region showing the 3D electrode interface in the sample channel.

FIG. 5 is a flowchart that describes a method for exchanging a buffer in which a heterogeneous set of target cells included in a biological sample are suspended.

### DETAILED DESCRIPTION

Microfluidic cell enrichment by dielectrophoresis (DEP), based on biophysical and electrophysiology phenotypes, can involve cells being resuspended from their physiological media into a lower conductivity buffer, such as for enhancing force fields and enabling the dielectric contrast needed for separation and vice versa for long-term maintenance of the collected separated fraction. To help ensure that certain sensitive cells are not disrupted, such as via centrifugation methods for resuspension, the present inventors have conceived of an on-chip microfluidic approach for swapping cells into media tailored for dielectrophoresis. This approach can facilitate transfer of cells from physiological media into a relatively lower conductivity media, e.g., by using tangential flows of low media conductivity at a relatively higher flow rate versus sample flow, such as to help promote ion diffusion over the length of a main channel of a microfluidic structure.

Biological samples used in basic research and in clinical diagnostic settings can involve a degree of heterogeneity due to cellular subpopulations. Such heterogeneity can present challenges towards correlating specific cell types and markers to functional outcomes of interest, e.g., to disease onset and progression. Approaches for addressing this heterogeneity can involve using antibody receptors to surface markers that identify each cell type, such as for sorting cells of interest after fluorescent staining or magnetic functionalization. A challenge with such approaches is that these sample preparation operations can be time consuming, require costly chemicals, introduce a degree of selection bias and cause sample dilution to inhibit the enrichment level achievable for fractional subpopulations. Furthermore, characteristic cellular surface markers are often unavailable for key biological functions, such as cancer metastasis, stem cell differentiation lineage and immune cell activation. Hence, label-free cellular separations based on their inherent biophysical properties can present advantages, such as microfluidic systems which can present a dynamic platform with sufficient force fields for controlled deflection of a particular cell type from heterogeneous samples. Platforms for cell separations based on biophysical differences in size, shape, deformability, and electrical properties can be particularly advantageous to help quantify heterogeneity of cellular systems.

Another approach for cell separation or differentiation based on subcellular differences in physiology, such as electrical size, membrane morphology and cytoplasmic organization, involves dielectrophoresis (DEP). Here, microfluidic systems can be configured with spatial field non-uniformities, such as for translation of polarized cells towards the high field region by positive DEP (pDEP) or for translation of cells away from the high field due to polarization of the surrounding media. In this manner, DEP can be applied to distinguish or isolate circulating tumor cells, stem cell progenitors, cells based on their mitochondrial phenotype, bacterial strain discrimination, and isolation of secreted exosomes. The DEP trapping force (FDEP) for manipulating cells in a biological sample depends on the dielectric contrast between the cell and its surrounding media, at specific frequencies of the applied electric field. At typical operating frequencies for DEP (e.g., <10 Megahertz (MHz), the dielectric contrast is determined by a difference in conductivity of the cell interior to that of the suspending media. For biological cells that generally have an interior conductivity within a range of about 5000-15,000 μS/cm range, significant levels of pDEP can only be observed within media of conductivity in the 500-1000 μS/cm range and under electric fields in the >0.1 MHz range. While nDEP is possible in media of higher conductivity under electric fields in the <0.1 MHz range, the degree of dielectric contrast to differentiate cells based on their DEP behavior is insubstantial and electrothermal flows arising from Joule heating due to electric fields within high conductivity media can further lower the resolution of the separations. Thus, biological cells in a sample can necessitate, in certain instances, being swapped, e.g., from their culture media that typically possess relatively higher conductivity levels to the optimal buffer media for DEP, which can be at or near a lower conductivity. One approach to swapping biological cells from their culture media involves an off-chip technique requiring sensitive cells to be repeatedly centrifuged. This can involve user intervention can increase the time spent by certain sensitive cells outside of their culture media. A challenge with this technique involves a loss of cell viability post-DEP separation, which can occur due to the sample preparation steps in centrifugation.

This document describes a microfluidic structure for swapping biological samples, e.g., from a cell culture medium or in vivo biopsy, to a measurement or isolation buffer. Such a microfluidic structure can help streamline a buffer swapping process while avoiding flow dispersion and dilution of cells. A single-cell or near single-cell streamline can be formed that facilitates ion diffusion. One or more focusing flows can be applied to a biological cell to help form the streamline. Such focusing flows can, effectively, increase the velocity of the cells traveling through the microfluidic device, but limit acceleration under a threshold that would exceed the desired conditions for downstream electrical measurement or separation. This approach can be especially advantageous for cell cultures or biopsies of small sample sizes or for delicate cells that would not be able to survive the traditional process of centrifugation and resuspension.

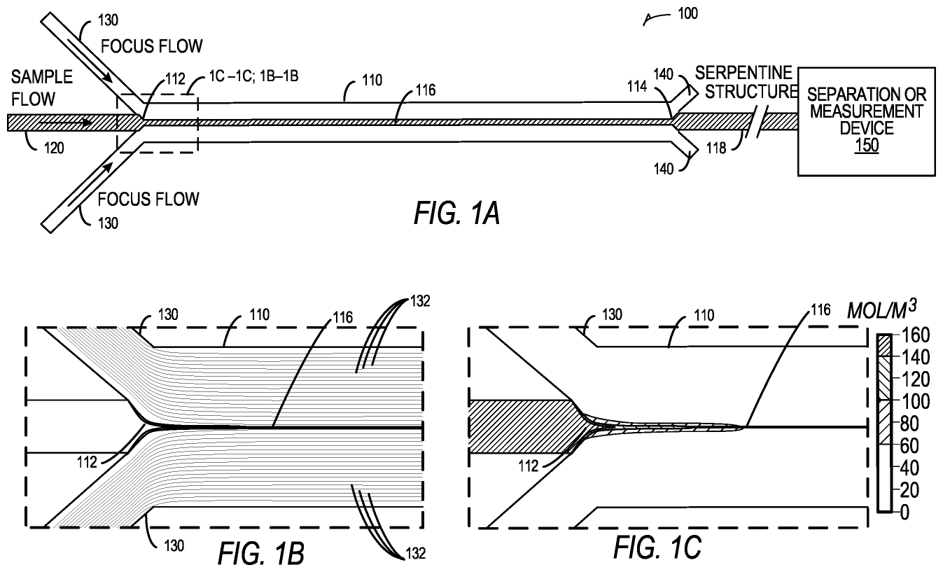

FIG. 1 depicts an example of a microfluidic structure for exchanging a buffer of target cells such as for an on-chip biological separation and measurement. The microfluidic structure 100 can include a main channel 110 defining an inlet 112 and an outlet 114, the main channel 110 for receiving a biological sample including a heterogeneous set of target cells. The microfluidic structure 100 can also include a sample insertion channel 120 fluidically connected to the inlet 112 and flow through the sample insertion channel 120 can be controlled such as to establish or adjust a first flow rate of the target cells, suspended in a first buffer, at the inlet.

The microfluidic structure 100 can include at least one focusing flow channel 130 feeding a main channel 110. Focusing flow supplied by the at least one focusing flow channel 130 can be controlled such as to establish or adjust a second flow rate of a second buffer. For example, the sample insertion channel 120 can be sized and shaped at a smaller depth than that of the focusing flow channel 130 to increase an interfacial area between the central region 116 and the second buffer. Herein, the central region 116 can also be referred to as a streamline 116 Also, the microfluidic structure 100 can include respective nozzle structures at an outlet of the focusing flow channel 130 and feeding the main channel 110. Here, an individual respective nozzle structure can include a tapered profile such as to help establish the focusing flow in the main channel 110. In an example, the first buffer can possess a conductivity level of about 15,000 microsiemens per centimeter (μS/cm) and the second buffer can possess be at or near about 500-fold lower conductivity (e.g., 500-500 μS/cm).

In an example, the second flow rate can be established or adjusted to promote or optimize ion diffusion between the first buffer and the second buffer. For example, a higher flow rate of the second buffer, relative to the first buffer, can enhance the probability of ion transfer. A velocity gradient established at the boundary can inhibit a dispersion of the target cells away from the central region. For example, the second flow rate can be established at a ratio within a range of about 3:1 and about 10:1 relative to the first flow rate. Here, the velocity gradient can help suppress bulk convection between the first buffer and the second buffer during ion diffusion in the channel 140 of the microfluidic structure 100.

In an example, the microfluidic structure 100 can also include a serpentine structure 118 fluidically connected to the outlet of the main channel 110, the serpentine structure for introducing a flow resistance at the outlet of the main channel 110 to control a velocity of the target cells in the central region before a downstream operation. For example, the serpentine structure 118 can define at least one bend and an internal channel that joins an inlet and an outlet of the main channel 110 at a cross section. Here, an internal channel of the serpentine structure 118 can be sized and shaped such as to increase a flow resistance to the target cells. Also, the microfluidic structure can include at least one side effluent channel 140,

also described herein as at least one flanking buffer outlet 140. The at least one side effluent channel 140 can be sized and shaped such as to collect an excess flow of the second buffer. For example, the at least one side effluent channel 140 can be sized and shaped such as to divert a flow of buffer solution away from the central region at or near the outlet of the main channel 110. In an example, the side effluent channel 140 can be configured to have a larger depth than that of the main channel 110 such that the side effluent channel 140 is positioned at a greater depth than the side effluent channel 140 relative to the main channel 110. For example, the side effluent channel 140 can be made longer than the main channel 110, at least from the outlet of the main channel 110 to the outlet of the side effluent channel 140.

In an example, the microfluidic device can include at least one separation or measurement device 150. Here, the at least one separation or measurement device 150 can be positioned at a downstream location relative to the serpentine structure 118. The at least one separation or measurement device 150 can facilitate separation of the target cells from the biological sample. For example, the at least one separation or measurement device 150 can include a dielectrophoretic (DEP) structure for separating target cells from the biological sample based on biophysical differences between the target cells. Also, the at least one separation or measurement device 150 can include a measurement structure for measuring a characteristic of the target cells, e.g., a cell size, shape, electrical impedance, intracellular concentration, etc.

FIG. 1B is a detailed view of the inlet 112 of the main channel 110 depicted in FIG. 1A. Here, orientation of the sample insertion channel and at least one focusing flow channel can facilitate a sheeting flow. Here, the sheeting flow is created by nature of the focusing flow channels flowing at a velocity much higher than the sample insertion channel. Such an orientation can also maximize an interfacial area between the second buffer of the focusing flow and first buffer of the biological sample flow. The sheeting flow can also help form a streamline in the central region 116, a laminar flow of the second buffer (depicted by lines 132) in the main channel 110, and a laminar flow of the first buffer in the sample insertion channel 120.

FIG. 1C is another detailed view of the inlet 112 of the main channel 110 depicted in FIG. 1A. The microfluidic structure 100 can facilitate diffusion-driven ion migration through a region of the sample flow. Such a structure can be sized and shaped to provide flow rates which help prevent mixing and convection of molecules between the first buffer and the second buffer, as well as to provide a requisite amount of time for an electric field to act upon a cell streamline in the central region 116. To do this, a second buffer solution with a higher velocity than the sample flow is used. As depicted in the chart in FIG. 1C, this second buffer solution (depicted as a lower concentration substance in moles per meter cubed (mol/m³)) surrounds the sample flow (depicted as a higher concentration substance in mol/m³) and creates a confined region for diffusion and ion migration to occur. The length of the region for such diffusion can be within a range of about 1 millimeter to about 1 centimeter. Also, the velocity of the second buffer solution can be within a range of about 0.01 milliliters per second to about 0.1 milliliters per second, while the velocity of the sample flow can be within a range of about 0.001 milliliters per second to about 0.01 milliliters per second.

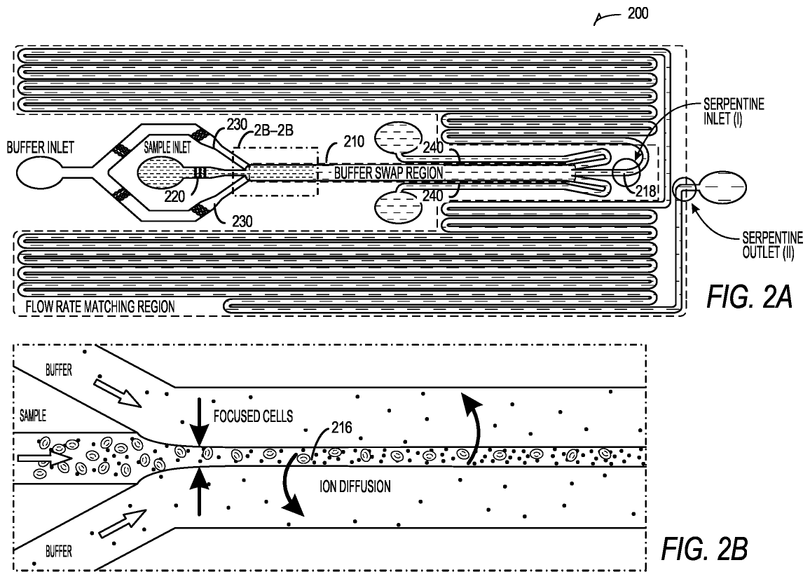

FIG. 2A and FIG. 2B depict an example of a microfluidic device for exchanging a buffer of target cells such as for on-chip biological separation and measurement. The microfluidic device 200 can be substantially similar to the microfluidic structure 100 of the example of FIG. 1A, FIG. 1B, and FIG. 1C. The components, structures, configuration, functions, etc. of the microfluidic device 200 can therefore be the same as or substantially similar to that described in detail above with reference to the microfluidic structure 100. Similar to that described above, the microfluidic device 200 can facilitate an efficient diffusion of a sample flow with second buffer supplied by at least one focusing flow, e.g., while impeding travel of particles between the first and second buffers by convection. The microfluidic device 200 can include a sample insertion channel 220 and at least one focusing flow channel 230. The sample insertion channel 220 can be formed at a relatively smaller depth, while the at least one focusing flow channel 230 can be formed at a relatively larger depth. This can facilitate flow such that the second buffer, when supplied by an interfacing plurality of focusing flows, can “hug” around the sample insertion channel 220 and can increase an interfacial area that is available for ion diffusion. Also, the at least one focusing flow channel 230 can provide the second buffer flowing at a higher velocity than the first buffer flowing through the sample insertion channel 220. To further facilitate diffusion, the microfluidic device can include at least one flow constriction or nozzle structure to help establish or adjust a velocity of the second buffer solution and establish or adjust a length over which the ion diffusion, between the first buffer and the second buffer, occurs. As depicted in FIG. 2A, the microfluidic device 200 can involve flow focusing of cells at the center of a main channel 210 by tangential flows, such as involving ion diffusion at the edges of the main channel 210, e.g., for enabling on-chip media swap of cells from their culture media to a lower conductivity media (e.g., ˜100-fold lower). In an example, the microfluidic device 200 can include a main channel that is about 2 centimeters (cm) in length, about 1500 micrometers (μm) in width and about 50 μm in depth. Such a sizing and shaping of the main channel can facilitate high laminarity to help minimize dispersion of cell streamlines, such that cells can be exchanged from physiological media to that of low conductivity media, as advantageous for downstream DEP deflection.

The collection region for cells in the swapped buffer can include a sample outlet 218, e.g., sized at a relatively larger width than that of the flow-focused cell streamlines in the central region 216. Also, the sample outlet 218 can provide a hydrodynamic resistance that is relatively higher than that of the two flanking excess buffer outlets or effluent channels 240.

For example, the sample outlet 218 can include or be fluidically coupled to a serpentine structure. In an example, the serpentine structure can be formed at a length that is within a range of about 90 times and about 110 times the length of an individual effluent channel 240. Such a configuration can help reduce a flow rate and flow dispersion of the cell streamline in the central region 216 while facilitating removal of any excess of the second buffer. Here, the cells in the sample can be collected without significant dilution and at reduced velocities that support downstream DEP deflection at a desired separation throughput. The enhanced net hydrodynamic resistance of the sample outlet 218 can also facilitate tolerance to certain external flow disturbances or modulations. The collected cells in the swapped buffer pass onward to an adjoining microchannel for in-line dynamic DEP at the same flow rate, through connective tubing between the sample outlet of the buffer swap stage and the sample inlet of the DEP stage.

In an example, as depicted in FIG. 2B, red blood cells (RBCs) can be received in the sample inlet and can be funneled or focused towards a central region 216 of the microfluidic device 200. The microfluidic device 200 can facilitate transfer of the RBCs from a media of 1×PBS (phosphate buffered saline) at about 15000 μS/cm conductivity to a buffer with a media conductivity of about 175 μS/cm. Here, a collected sample at a sample outlet can exhibit minimal dilution (e.g., the sample diluted from a concentration within a range of about 3×10\hat{ }8 and 0.3 billion cells/mL to a concentration within a range of about 1×10\hat{ }8 and 0.1 billion cells/mL). A heterogenous set of cells collected at the sample outlet can support in-line negative dielectrophoresis (nDEP), e.g., at 30 kHz, and positive dielectrophoresis (pDEP), e.g., at 1 MHz.

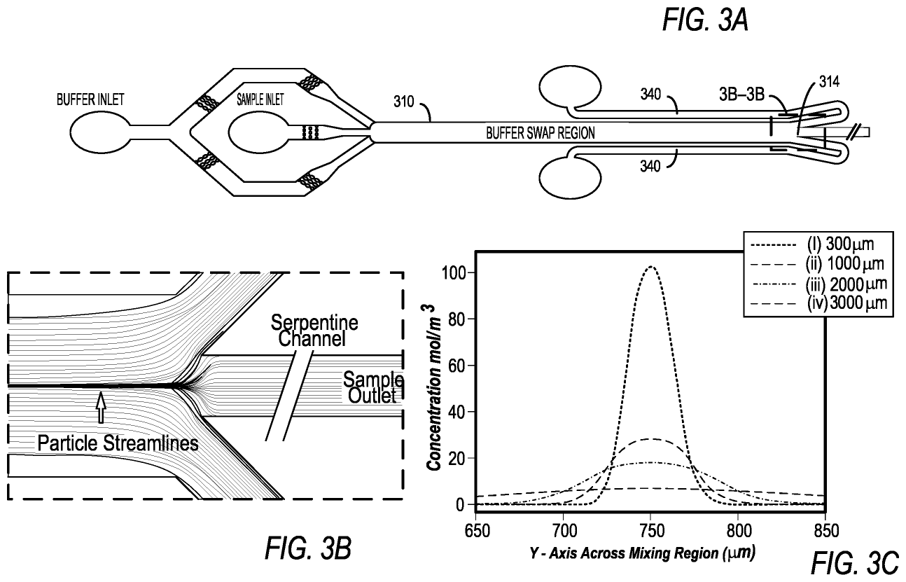

FIG. 3A & FIG. 3B depict another example of a microfluidic device for exchanging a buffer of target cells such as for on-chip biological separation and measurement. The microfluidic device 300 can be substantially similar to the microfluidic structure 100 of the example of FIG. 1A, FIG. 1B, and FIG. 1C and the microfluidic device 200 of the example of FIG. 2A and FIG. 2B. The components, structures, configuration, functions, etc. of the microfluidic device 300 can therefore be the same as or substantially similar to that described in detail above with reference to the microfluidic device 200 and the microfluidic structure 100.

The microfluidic device 300 can include flanking outlets or effluent channels 340, through which the flow resistance of the outlet 314 of the main channel 310 is adjusted such as to help offset an acceleration of the target cells applied by the focusing flows. The effluent channels 340 can also help slow the target cells such as to allow a sufficient amount of time for diffusion of ions. Additionally, the flow resistance of the effluent channels 340 can be greater than the flow resistance in the main channel 310 such as to help impede cells from entering the effluent channels 340. As depicted in the detailed view in FIG. 3B, streamlines for cells and second buffer from the focusing flow indicate differences in flow velocity of the central streamline as compared to the effluent channels 340 from the buffer swap stage. Such a difference in velocity at the outlet 314 of the main channel 310 can be in part due to the excess hydrodynamic resistance from the serpentine channel after the central outlet.

FIG. 3C shows a chart depicting an example of a modeling exercise used to calculate the desired length of the microfluidic device 300 such as to achieve equalized media conductivity and inhibit cell dispersion. In an example, certain parameters of the cells, such as size, type and phenotypic characteristics, to can be modeled to help determine an optimal buffer system. Such a model can also factor certain differences between the conductivities of the cells' original media and the media in which the electrical measurement or separation is conducted. Here, the model can help determine parameters to balance between optimizing the flow resistance of the central outlet and preventing cells from breaking out of the central outlet and into the flanking outlets. As depicted in FIG. 3C, ion concentration profiles can span a width of the microchannel along progressively longer mixing lengths within a main channel 310 of the microfluidic device 300: e.g., (i) 300 micrometers (μm), (ii) 1000 μm, (iii) 2000 μm, and (iv) 3000 μm. In an example, the concentration of cells is high in the center and low on the edges. At lengths greater than 3000 microns (3 millimeters), the concentrations can move towards being relatively equalized. To help ensure cells are not carried away by mixing or convection, particle streamlines can be used to calculate the threshold level of resistance needed to slow the cells down without causing them to deflect to the effluent channels 340. The serpentine structure also facilitates an increase in the flow resistance of the central outlet while allowing the buffer to exit quickly through the effluent channels 340.

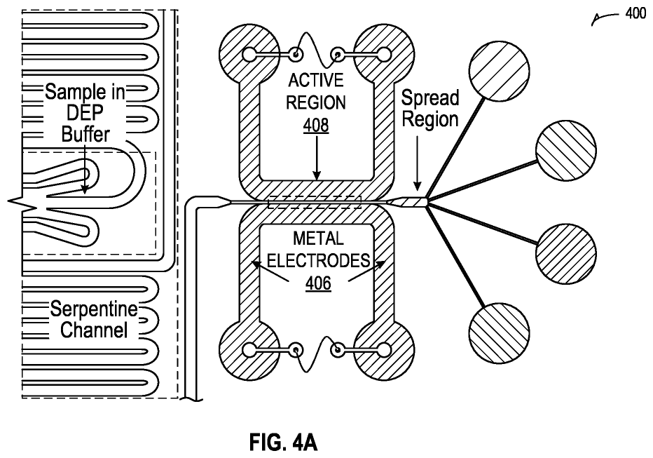

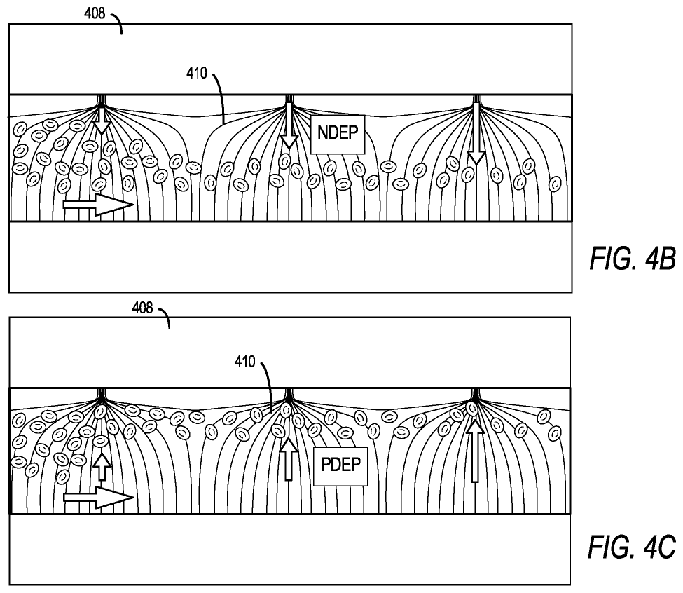

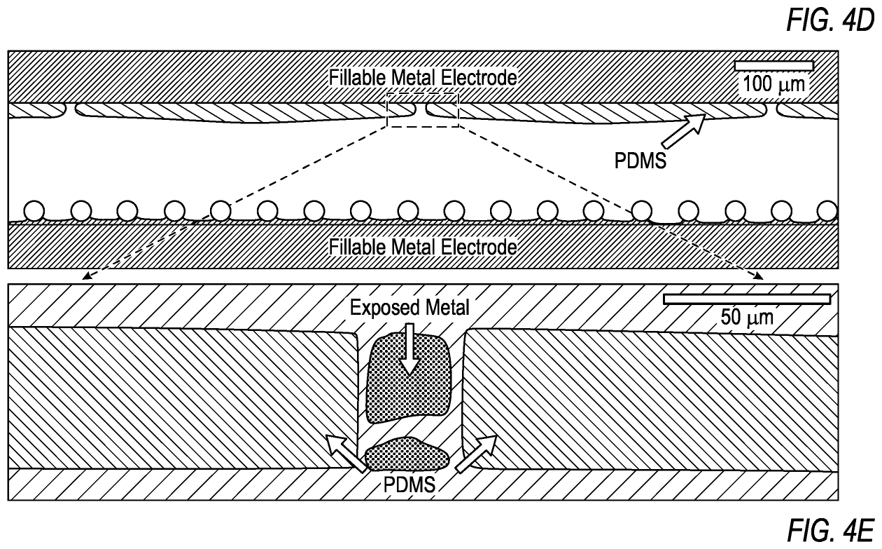

FIG. 4A, FIG. 4B, and FIG. 4C depict an example of a microfluidic device for separating particles in a sample based on biophysical properties. The microfluidic device 400 can be substantially similar to the microfluidic structure 100 of the example of FIG. 1A, FIG. 1B, and FIG. 1C, the microfluidic device 200 of the example of FIG. 2A and FIG. 2B, and the microfluidic device 300 of the example of FIG. 3A and FIG. 3B. The components, structures, configuration, functions, etc. of the microfluidic device 300 can therefore be the same as or substantially similar to that described in detail above with reference to the microfluidic device 300, the microfluidic device 200, and the microfluidic structure 100.

FIG. 4A shows a connection from buffer swap region to a DEP device region through a serpentine channel to modulate hydrodynamic resistance. In an example, a microfluidic device 400 can include at least one focusing flow at or near a sample input to fix its position with respect to the sequential field non-uniformities for separation. Metal electrodes 406 can be included in the device to generate an electric field and a sample can be loaded into the device, resulting in a sample flow. The sample flow can be directed through an active region 408, which can include a series of electrical fields of varying strengths. An individual electric field can cause particles to move toward or away from an individual electrode, resulting in a separation of particles based on their biophysical properties, such as size and/or charge. As depicted in FIG. 4A, FIG. 4B, and FIG. 4C electrical fields depicted by field lines 410 can cause particle deflection from initial focused position by negative DEP (nDEP) (FIG. 4B) and positive DEP (pDEP) (FIG. 4C). A sensing system can be included in the device to detect the particles and output a signal. The output signal can be used to adjust the electric field or to control the flow, resulting in a separation of particles based on their biophysical properties.

FIG. 4D and FIG. 4E depict an electrical DEP separation or electrical sensing device. The electrode architecture across a sample channel can introduce sequential field non-uniformities. FIG. 4E depicts an expanded view of the orifice region showing the 3D electrode interface in the sample channel. For on-chip monitoring of the media conductivity after the buffer swap by impedance sensing, the microchannel can be integrated with sidewall electrodes that extend over the channel depth. Certain planar electrode strategies can involve a limited spatial extent over channel depth and can be suboptimal for detecting media conductions under microfluidic flow focusing or diffusional dilution. Instead, the electrode architecture depicted in FIG. 4D and FIG. 4E can involve co-fabrication strategies, wherein a liquified metal electrode (e.g., Field's metal) is filled in an electrode channel that is self-aligned to a sample channel, can be used to create sidewall electrodes in a microchannel. In an example, such as to restrict the liquid metal filling to the electrode channel, a confined depth can be included. For example, the electrode in the microchannel can include multilayer SU8 photoresist on silicon followed by Polydimethylsiloxane (PDMS) micromolding. More specifically, the electrode in the microchannel can include a single layer patterning of SU-8 photoresist by photolithography on an approximately 4″ width silicon wafer to approximately 50 μm depth. In an example, a step height between the electrode and sample channels of the device can be configured such that the sensing area is maximized while maintaining a uniform topography over the length of the sensing area. In an example, the electrical separation or electrical measurement device can include or use a sidewall electrode of substantially uniform topography over lengths on the order of >100 μm.

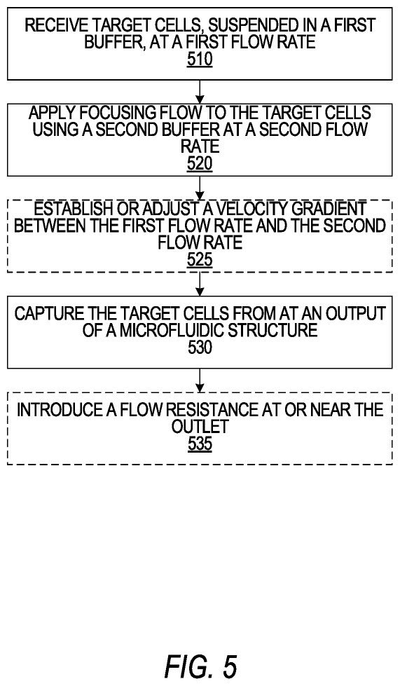

FIG. 5 is a flowchart that describes a method for exchanging a buffer in which a heterogeneous set of target cells included in a biological sample are suspended.

At 510, the method can include receiving a biological sample comprising target cells, at an inlet of a microfluidic structure including flowing the target cells. Here, the target cells can be suspended in a first buffer and flowing at a first flow rate within a main channel of the microfluidic structure. In an example, the biological sample can include less than 20,000 target cells within microliter scale volumes. In an example, the target cells can include at least one of stem cells, immune cells, or cancer cells. In an example, the exchanging the heterogenous set of the target cells can be suspended, from the first buffer towards the second buffer, occurs without centrifugation.

At 520, the method can include applying at least one focusing flow to the biological sample. For example, the at least one focusing flow can be a set of interfacial focusing flows to the biological sample. The at least one focusing flow can be applied using a second buffer at a second flow rate, such as to establish a boundary defining a central region containing the target cells within the main channel, and promoting ion diffusion between the first buffer and the second buffer.

Optionally, at 525, the method can include establishing or adjusting a velocity gradient between the first flow rate and the second flow rate to inhibit dispersion of the target cells away from the central region. A velocity gradient established at the boundary can inhibit dispersion of the target cells away from the central region and supports its downstream biological separation and measurement. In an example, the second flow rate can be established at a ratio between 3:1 and 10:1 relative to the first flow rate. In an example, the velocity gradient suppresses bulk convection between the first buffer and the second buffer during ion diffusion in the channel of the microfluidic structure.

At 530, the method can include capturing the target cells from the central region at an output of a microfluidic structure. A heterogeneous set of target cells included in a biological sample can be suspended, such as for an on-chip biological separation and measurement task. In an example, the microfluidic structure can define respective nozzle structures feeding the main channel, the respective nozzle structures having a tapered profiled to establish the focusing flow in the main channel. The microfluidic structure can also define side effluent channels to divert a flow of buffer solution away from the central region at or near the output of the microfluidic structure.

Optionally, at 535, the method can include introducing a flow resistance at an outlet of the main channel to control a velocity of the target cells in the central region before a downstream operation. In an example, the method can include performing electrical measurement or electrical separation of the central region of target cells using at least one of electrophoresis, dielectrophoresis, electrochemical measurement, or impedance measurement of cells or media.

The following, non-limiting examples, detail certain aspects of the present subject matter to solve the challenges and provide the benefits discussed herein, among others.

Example 1 is a method for exchanging a buffer in which a heterogeneous set of target cells included in a biological sample are suspended, such as for an on-chip biological separation and measurement task, the method comprising: receiving a biological sample comprising the target cells, at an inlet of a microfluidic structure including flowing the target cells, suspended in a first buffer, at a first flow rate within a main channel of the microfluidic structure; applying a set of interfacial focusing flows to the biological sample using a second buffer at a second flow rate to establish a boundary defining a central region containing the target cells within the main channel, and promoting ion diffusion between the first buffer and the second buffer; and capturing the target cells from the central region at an output of a microfluidic structure; wherein a velocity gradient established at the boundary inhibits dispersion of the target cells away from the central region and supports its downstream biological separation and measurement.

In Example 2, the subject matter of Example 1 includes, wherein the microfluidic structure defines respective nozzle structures feeding the main channel, the respective nozzle structures having a tapered profiled to establish the focusing flow in the main channel.

In Example 3, the subject matter of Examples 1-2 includes, wherein the microfluidic structure defines side effluent channels to divert a flow of buffer solution away from the central region at or near the output of the microfluidic structure.

In Example 4, the subject matter of Examples 1-3 includes, establishing or adjusting a velocity gradient between the first flow rate and the second flow rate to inhibit dispersion of the target cells away from the central region.

In Example 5, the subject matter of Examples 1-4 includes, wherein the second flow rate is established at a ratio between three to one and ten to one relative to the first flow rate.

In Example 6, the subject matter of Examples 1-5 includes, wherein the velocity gradient suppresses bulk convection between the first buffer and the second buffer during ion diffusion in the channel of the microfluidic structure.

In Example 7, the subject matter of Examples 1-6 includes, wherein the biological sample includes less than twenty thousand target cells within microliter scale volumes.

In Example 8, the subject matter of Examples 1-7 includes, wherein the target cells include at least one of stem cells, immune cells, or cancer cells.

In Example 9, the subject matter of Examples 1-8 includes, wherein the exchanging the heterogenous set of the target cells are suspended, from the first buffer towards the second buffer, occurs without centrifugation.

In Example 10, the subject matter of Examples 1-9 includes, introducing a flow resistance at an outlet of the main channel to control a velocity of the target cells in the central region before a downstream operation.

In Example 11, the subject matter of Examples 1-10 includes, performing electrical measurement or electrical separation of the central region of target cells using at least one of electrophoresis, dielectrophoresis, electrochemical measurement, or impedance measurement of cells or media.

Example 12 is a microfluidic structure for exchanging a buffer in which a heterogeneous set of target cells included in a biological sample are suspended, such as for an on-chip biological separation and measurement task, the microfluidic structure comprising: a main channel defining an inlet and an outlet, the main channel configured to receive a biological sample comprising the target cells, at the inlet; a sample insertion channel fluidically connected to the inlet and configured to establish or adjust a first flow rate of the target cells, suspended in a first buffer, at the inlet; a focusing flow channel feeding the main channel and configured to establish or adjust a second flow rate of a second buffer, wherein the second buffer is applied to establish a boundary defining a central region containing the target cells within the main channel, the second flow rate established or adjusted to promote ion diffusion between the first buffer and the second buffer; and at least one side effluent channel to collect an excess flow of the second buffer; wherein a velocity gradient established at the boundary inhibits a dispersion of the target cells away from the central region.

In Example 13, the subject matter of Example 12 includes, wherein the sample insertion channel is sized and shaped at a smaller depth than that of the focusing flow channel to increase an interfacial area between the central region and the second buffer.

In Example 14, the subject matter of Examples 12-13 includes, respective nozzle structures at an outlet of the focusing flow channel and feeding the main channel, the respective nozzle structures having a tapered profiled to establish the focusing flow in the main channel.

In Example 15, the subject matter of Example 14 includes, wherein the respective nozzle structures are configured to establish or adjust a velocity gradient between the first flow rate and the second flow rate to inhibit dispersion of the target cells away from the central region.

In Example 16, the subject matter of Examples 12-15 includes, wherein the at least one side effluent channel is configured to divert a flow of buffer solution away from the central region at or near the outlet of the main channel.

In Example 17, the subject matter of Examples 12-16 includes, wherein the second flow rate is established at a ratio between three to one and ten to one relative to the first flow rate.

In Example 18, the subject matter of Examples 12-17 includes, wherein the velocity gradient suppresses bulk convection between the first buffer and the second buffer during ion diffusion in the channel of the microfluidic structure.

In Example 19, the subject matter of Examples 12-18 includes, a serpentine structure fluidically connected to the outlet of the main channel, the serpentine structure for introducing a flow resistance at the outlet of the main channel to control a velocity of the target cells in the central region before a downstream operation.

Example 20 is a method comprising: receiving a biological sample comprising target cells, at an inlet of a microfluidic structure including flowing the target cells, suspended in a first buffer, at a first flow rate within a main channel of the microfluidic structure; applying at least one focusing flow to the biological sample using a second buffer at a second flow rate to establish a boundary defining a central region containing the target cells within the main channel, and promoting ion diffusion between the first buffer and the second buffer; and capturing the target cells from the central region at an output of a microfluidic structure; wherein a velocity gradient established at the boundary inhibits dispersion of the target cells away from the central region and supports its downstream biological separation and measurement.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims.

Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for exchanging a buffer in which a heterogeneous set of target cells included in a biological sample are suspended, prior to or following an on-chip biological separation and measurement task, the method comprising:
receiving a biological sample comprising the target cells, at an inlet of a microfluidic structure including flowing the target cells, suspended in a first buffer, at a first flow rate within a main channel of the microfluidic structure;
applying a set of interfacial focusing flows to the biological sample using a second buffer at a second flow rate to establish a boundary defining a central region containing the target cells within the main channel, and promoting ion diffusion between the first buffer and the second buffer; and
capturing the target cells from the central region at an output of a microfluidic structure;
wherein a velocity gradient established at the boundary inhibits dispersion of the target cells away from the central region.

2. The method of claim 1, wherein the microfluidic structure defines respective nozzle structures feeding the main channel, the respective nozzle structures having a tapered profiled to establish the focusing flow in the main channel.

3. The method of claim 1, wherein the microfluidic structure defines side effluent channels to divert a flow of buffer solution away from the central region at or near the output of the microfluidic structure.

4. The method of claim 1, comprising establishing or adjusting a velocity gradient between the first flow rate and the second flow rate to inhibit dispersion of the target cells away from the central region.

5. The method of claim 1, wherein the second flow rate is established at a ratio between 3:1 and 10:1 relative to the first flow rate.

6. The method of claim 1, wherein the velocity gradient suppresses bulk convection between the first buffer and the second buffer during ion diffusion in the channel of the microfluidic structure.

7. The method of claim 1, wherein the biological sample includes less than 20,000 target cells within microliter scale volumes.

8. The method of claim 1, wherein the target cells include at least one of stem cells, immune cells, or cancer cells.

9. The method of claim 1, wherein the exchanging the heterogenous set of the target cells are suspended, from the first buffer towards the second buffer, occurs without centrifugation.

10. The method of claim 1, comprising introducing a flow resistance at an outlet of the main channel to control a velocity of the target cells in the central region before a downstream operation.

11. The method of claim 1, comprising performing electrical measurement or electrical separation of the central region of target cells using at least one of electrophoresis, dielectrophoresis, electrochemical measurement, or impedance measurement of cells or media.

12. A microfluidic structure for exchanging a buffer in which a heterogeneous set of target cells included in a biological sample are suspended, prior to or following an on-chip biological separation and measurement task, the microfluidic structure comprising:
a main channel defining an inlet and an outlet, the main channel configured to receive a biological sample comprising the target cells, at the inlet;
a sample insertion channel fluidically connected to the inlet and configured to establish or adjust a first flow rate of the target cells, suspended in a first buffer, at the inlet;
a focusing flow channel feeding the main channel and configured to establish or adjust a second flow rate of a second buffer, wherein the second buffer is applied to establish a boundary defining a central region containing the target cells within the main channel, the second flow rate established or adjusted to promote ion diffusion between the first buffer and the second buffer; and
at least one side effluent channel to collect an excess flow of the second buffer;
wherein a velocity gradient established at the boundary inhibits a dispersion of the target cells away from the central region.

13. The microfluidic structure of claim 12, wherein the sample insertion channel is sized and shaped at a smaller depth than that of the focusing flow channel to increase an interfacial area between the central region and the second buffer.

14. The microfluidic structure of claim 12, comprising respective nozzle structures at an outlet of the focusing flow channel and feeding the main channel, the respective nozzle structures having a tapered profiled to establish the focusing flow in the main channel.

15. The microfluidic structure of claim 14, wherein the respective nozzle structures are configured to establish or adjust a velocity gradient between the first flow rate and the second flow rate to inhibit dispersion of the target cells away from the central region.

16. The microfluidic structure of claim 12, wherein the at least one side effluent channel is configured to divert a flow of buffer solution away from the central region at or near the outlet of the main channel.

17. The microfluidic structure of claim 12 wherein the second flow rate is established at a ratio between 3:1 and 10:1 relative to the first flow rate.

18. The microfluidic structure of claim 12, wherein the velocity gradient suppresses bulk convection between the first buffer and the second buffer during ion diffusion in the channel of the microfluidic structure.

19. The microfluidic structure of claim 12, comprising a serpentine structure fluidically connected to the outlet of the main channel, the serpentine structure for introducing a flow resistance at the outlet of the main channel to control a velocity of the target cells in the central region before a downstream operation.

20. A method comprising:
receiving a biological sample comprising target cells, at an inlet of a microfluidic structure including flowing the target cells, suspended in a first buffer, at a first flow rate within a main channel of the microfluidic structure;
applying at least one focusing flow to the biological sample using a second buffer at a second flow rate to establish a boundary defining a central region containing the target cells within the main channel, and promoting ion diffusion between the first buffer and the second buffer; and
capturing the target cells from the central region at an output of a microfluidic structure;
wherein a velocity gradient established at the boundary inhibits dispersion of the target cells away from the central region.
